(19) United States
(12) Patent Application Publication
(10) Pub. No.: US 2004/0014818 A1
(43) Pub. Date: Jan. 22, 2004

(54) Bactericidal preparation

(76) Inventors: Betty Boeck, Wolongong, NSW (AU); Harry Boeck, Fairy Meadow (AU)

Correspondence Address: MOLINS AND CO., LEVEL 25 CHIFLEY TOWER, SYDNEY, 2000, AU

(21) Appl. No.: 10/221,997

(22) PCT Filed: Mar. 14, 2001
(86) PCT No.: PCT/AU01/00275

(30) Foreign Application Priority Data
Mar. 20, 2000 (AU) PQ 6323

(51) Int. Cl. A61K 31/05; A61K 31/015

(52) U.S. Cl. 514/731; 514/763

(57) ABSTRACT

A bactericidal preparation in the form of a solution, cream or ointment is disclosed. The preparation comprises a liquid compounded from photosynthesized hydrocarbons, isolates from hydrocarbons, 2-hydroxy-1-isopropyl-4-methyl-benzene (thymol) and butylated hydroxytoluene.

This invention relates to bactericidal preparations such as solutions and creams, in particular to topical solutions which may be applied to the skin and which exhibit penetrating action, whereby they are able to be absorbed into the skin to reach subdermal pathogens.

### BACKGROUND OF THE INVENTION

In order to fight pathogenic organisms on the skin and particularly in the body, chemical compounds under the general heading of “antibiotics” are administered by mouth (ingestion) or by injection and sometimes are applied to the surface of the skin in the form of creams or ointments.

Generally speaking, an antibiotic may be described as an organic substance which is produced by micro-organisms or has a molecular structure similar to naturally occurring substances and is capable at low concentration of inhibiting the growth of or destroying another micro-organism. Antibiotics have been isolated from numerous sources, but principally from bacteria (eg bacitracin, polymixin, gramicidan), actinomycetes (eg tetracycline, streptomycin, chloramphenicol) and fungi (eg penicillins, cephalosporins). Bacterial antibiotics are mostly polypeptides.

Many antibiotics however are unsuitable for therapeutic use, frequently because of their general toxicity or as a result of other drawbacks such as their inherent instability, inadequate solubility or malabsorption.

### OBJECT OF THE INVENTION

The object of the present invention is to provide an effective bactericidal preparation such as a solution or cream having “antibiotic” effectiveness, which may be topically applied to human skin, and which exhibits penetrating action, so that it is able to be absorbed in skin to reach subdermal pathogens. At the very least, the invention provides an alternative to presently known topical bactericidal compositions.

### DISCLOSURE OF THE INVENTION

According to the present invention there is provided a bactericidal preparation in the form of a solution, cream or ointment comprising a liquid compounded from photosynthesized hydrocarbons, isolates from hydrocarbons, 2-hydroxy-1-isopropyl-4-methyl-benzene (thymol) and butylated hydroxytoluene.

Preferably a solution according to the invention has the following ranges of composition:

| | |
|---|---|
| α-pinene | 1.9 to 2.0% |
| α-terpinene | 1.2 to 2.7% |
| limonene | 6.0 to 27.6% |
| ρ-cymene | 0.5 to 1.2% |
| 1,8 cineole | 2.5 to 25.6% |
| γ-terpinene | 2.8 to 6.5% |
| terpin-4-ol | 4.0 to 20.0% |
| γ-terpineol | 1.0 to 2.2% |
| neral | 2.2 to 9.2% |
| geranial | 1.5 to 11.5% |
| thymol | 0.3 to 2.0% |
| eugenol | 0.5 to 3.5% |
| butylated hydroxytoluene | 0.3 to 0.5% |
| Balance (insignificant variations | |
| in natural ingredients made up as | |
| required with ethanol) | |

Alternatively a preferred ointment formulation according to the invention has the following ranges of composition:

| | |
|---|---|
| Triclosan | 0.3 to 2.0% |
| Allantoin | 0.1 to 2.5% |
| Petrolatum | 40.0 to 70.0% |
| α-pinene | 1.9 to 2.0% |
| β-pinene | 1.2 to 2.7% |
| Myrcene | 0.5 to 1.2% |
| α-phellandrene | 0.3 to 0.4% |
| α-terpinene | 2.7 to 4.2% |
| limonene | 6.0 to 27.6% |
| β-phellandrene | 0.2 to 0.6% |
| 1,8 cineole | 2.5 to 25.6% |
| γ-terpinene | 2.8 to 6.5% |
| terpinolene | 1.0 to 4.0% |
| terpin-4-ol | 4.0 to 20.0% |
| α-terpineol | 1.0 to 2.2% |
| neral | 2.2 to 9.2% |
| geranial | 1.5 to 11.5% |
| thymol | 0.3 to 2.0% |
| eugenol | 0.5 to 3.5% |
| butylated hydroxytoluene | 0.3 to 0.5% |

Preferably a solution according to the invention has the following composition:

| | |
|---|---|
| α-pinene | 1.9% |
| α-terpinene | 1.2% |
| limonene | 25.5% |
| ρ-cymene | 1.0% |
| 1,8 cineole | 15.6% |
| γ-terpinene | 2.8% |
| terpin-4-ol | 20.0% |
| γ-terpineol | 2.2% |
| neral | 9.2% |
| geranial | 10.9% |
| thymol | 1.0% |
| eugenol | 2.4% |
| butylated hydroxytoluene | 0.5% |
| Balance (insignificant variations | |
| in natural ingredients made up as | |
| required with ethanol) | |

Alternatively a preferred ointment formulation according to the invention has the following composition:

| | |
|---|---|
| Triclosan | 1.0% |
| Allantoin | 1.0% |
| Petrolatum | 68.0% |
| α-pinene | 2.0% |
| β-pinene | 1.4% |
| Myrcene | 0.7% |
| α-phellandrene | 0.3% |
| α-terpinene | 2.7% |
| limonene | 27.6% |
| β-phellandrene | 0.2% |
| 1,8 cineole | 23.2% |
| γ-terpinene | 6.5% |
| terpinolene | 1.0% |
| terpin-4-ol | 10.4% |
| α-terpineol | 1.0% |
| neral | 6.3% |
| geranial | 7.5% |
| thymol | 0.6% |
| eugenol | 2.3% |
| butylated hydroxytoluene | 0.3% |

Preferably, undenatureded ethanol is added at varying levels to the solution, those levels being between 49.5% w-w and 97.4% w-w.

The ingredients are expressed in a percentage ratio by its proper chemical name. It will be understood that these percentages are able to be varied within a suitably expected ratio, arising from the use of several naturally occurring ingredients in the basic mix.

The compound solution, ointment or cream is thus the result of combining:

1. naturally occurring hydrocarbons,

2. specific isolates to increase the amount of specific constituents beyond that which may be found in nature,

3. butylated hydroxytoluene (2-6 di-tert-butyl-4-methylphenol).

The solution, ointment or cream according to the invention is not injected into the body or administered by mouth, rather the activity in the invention is by absorption through the skin. The ability of photosynthesized hydrocarbons to enter the body through skin is found in extensive literature and is the result of their molecular particle size and its polar structure.

It has been previously known that those substances comprising the invention can be associated in ethanol or fatty substances like oils. However, it has been discovered through actual trials that when the referenced hydrocarbons are combined with water potency is decreased and when dissolved in ethanol their potency is increased, since penetration into skin is limited by polar substances (water) they are therefore they are demonstrably less effective in destroying bacteria in deep-seated wounds.

It has also been found that certain constituents of naturally occurring hydrocarbons such as cineole, terpin-4-ol, terdenes, phenols and certain methylbenzenes are possessed of bactericidal properties. However, those constituents in combination powerful enough to be active in the very broad spectrum of pathogenic micro-organism are not found in naturally occurring substances in large enough concentrations by simple admixture of several existing naturally occurring compounds.

The invention therefore includes ingredients to boost the availability of certain individual substances to levels unobtainable in nature, and also includes a specific and highly substituted phenol. This compound is therefore not found in nature.

The preparation according to the invention acts on pathogens present in a nonspecific way by interrupting the metabolic uptake in the cell of pathogenic organisms and bursting the cell membrane. This activity is particularly noted in bacteria of the coccos genus, eg staphylococcus-aureus.

It should also be noted that present day “antibiotics” (as discussed above) are the result of isolates from numerous sources produced by micro-organisms or of a molecular structure similar to naturally occurring substances. The invention can thus be described as a topically applied “antibiotic”. Since the word “antibiotic” as coined by Selman Waksman in 1945 describes a chemical compound which was either bactericidal or bacteriostatic, the invention qualifies for that general description. However, the invention differs from an “antibiotic” in that its composition is of hydrocarbons rather than of a bacteria or fungi origin.

The preparation according to the invention is active in bacteria and fungi, and certain viruses. It has been found that the bactericidal solution according to the invention is able to destroy bacteria in the broadspectrum and is effective in genus staphylococcus resistant to methyciline and other forms of antibiotics.

The invention is thus useful in the control and treatment of bacterial infection in humans. Topical applications in low dosage, between 0.1 g and 0.3 g are known to be effective in penetrating a wound site. These findings are confirmed in in vitro tests and in vivo.

### EXAMPLE

A solution according to the preferred formulation above (or in some cases an equivalent cream formulation) was trialed on a number of patients.

The results are set out in the following table:

| | Injury & | | | |
|---|---|---|---|---|
| Patient | Treatment | Infection | Treatment | Outcome |
| Female 67 | Septic right | | Washed out, | Cleared at one |
| | ankle, treated | | solution into joint | week |
| | with Abs 2 | | | |
| | months | | | |
| Male 26 | Heroin addict, | Drained | Solution, 1 ml | Cleared in 48 |
| | septic right hip, | | into joint | hours |
| | staph aureus | | | |
| | ESR = 70 | | | |
| Male 24 | MVA, ORIF left | MRSA and | Solution into | Clear at two |
| | upper tibia Fx | discharging | wound via beads | weeks |
| | | sinus from | after R/O plate | |
| | | upper tibia. | | |
| | | Multiple | | |
| | | debridements & | | |
| | | antibiotics | | |
| Female 45 | TA wound | No organism | Cream applied | Wound healed at |
| | break-down, Pale | isolated | daily | one month |
| | and atrophic at 3 | | | |
| | months | | | |
| Male 31 | Open Calcaneal | Aeromonas | Wash out | Clear at two |
| | Fx, fall into | Hydrophillus | Solution sprayed | weeks |
| | Nepean River | | daily, ORIF | |
| Male 28 | MVA, ACI, | MRSA | Wash out, | Clear at six |
| | reconstruction | | solution into | weeks, solution |
| | | | knee at two | & antibiotics |
| | | | weeks, | needed as |
| | | | Rilampicin & | response was |
| | | | fucidin added | slow |
| Male 22 | Knee injury, ACL | Staph aureus | Solution into | Infection cleared |
| | reconstruction & | | knee for one | by 4 weeks, at 6 |
| | plateau Fx | | week & IV | months ESR & |
| | | | fluclox | CRP normal, no |
| | | | | signs of infection |
| Male 28 | 3B Fx right tibia, | MRSA from | Ext fix & | Knee superficial. |
| | MVA | wound | solution to | Wound cleared at |
| | | | wound for 8 | 4 weeks, ESR = 5, |
| | | | weeks with | CRP = 7 |
| | | | Debridement to | |
| | | | allow exchange | |
| | | | IM nall | |
| Female 42 | MVA, Fx tibia, | Non-union with | Excision of | OMB cleared at |
| | ORIF | sequestrum at 2 | sequestrum (2nd | 6 weeks, ESR = |
| | | years, pain & | time) BUT | 18 (at 3 months) |
| | | swelling, hot | solution added to | |
| | | bone scan, | bone at once | |
| | | Ciprofloxacin & | | |
| | | rifampicin for | | |
| | | two years | | |
| Male 43, | Lat llg | Staph aureus | Multiple | Wound clear & |
| diabetic | reconstruction of | | debridements, | closed at 3 |
| (non | ankle | | cream for two | weeks, cultures |
| insulin) | | | weeks | clear |
| Male 17 | MVA, Fx pelvis | MRSA from | IM nall removed, | Sinus dry at one |
| | & femur to | sinus, | cream for 6 days | week, cultures |
| | ORIF. Leter IM | Ciprofloxacin & | | clear at 8 weeks, |
| | nall femur for | rifempicin for | | ESR = 11, CRP = 11 |
| | non-union, | two years | | (at 8 |
| | discharging sinus | | | months) |
| Male 42 | Midfoot | MRSA after | Cream to | Healed after Vit |
| | Fx/dislocation | R/O, hardware | wound × | E cream added |
| | | 6 weeks. No | antibiotics | |
| Male 53 | Open Fx upper | MRSA | Solution percut | Cleared and ESR = 17 |
| | tibia & ankle | antibiotics & | via beads to inner | at 6 weeks |
| | | debridements | tibia | |
| | | for three years - | | |
| | | for amputation | | |

The preparation, whether solution, cream or ointment, according to the invention is effective in the very broad spectrum of gram stain negative and gram stain positive bacteria, fungi and certain viruses, it is also used in patients with infection by the resistant organism MRSA (Methicillin Resistant Staphylococcus aureus) commonly referred to as “Golden Staph”. It is known to be effective where contemporary antibiotics have failed and is not conducive to the emergence of true strain resistance. It is administered topically by doctors and orthopaedic surgeons to also treat chronic wound infections and is noted for its strong characteristic in accelerated clean wound healing.

Efficacy in the resistant organism, MRSA is consistent and persistent, and exhibits a 100% transfer of results from In Vitro to In Vivo. Allergic skin reactions are limited in 1-2% of cases to localised skin reddening which disappears in 1-2 days upon withdrawal of the treatment. The allergy appears to be specific to certain skin types, typically very fair skin. However, in those cases where reddening may occur an alternative treatment regime is available to the doctor in the post treatment of sensitive skin.

No systemic reactions have been recorded or expected.

The foregoing describes only some embodiments of the present invention, and modifications obvious to those skilled in the art can be made thereto without departing from the scope of the present invention.

1. A bactericidal preparation in the form of a solution, cream or ointment comprising a liquid compounded from photosynthesized hydrocarbons, isolates from hydrocarbons, 2-hydroxy-1-isopropyl-4-methyl-benzene (thymol) and butylated hydroxytoluene.

2. A bactericidal preparation according to claim 1, being a solution have the following ranges of composition by weight:

| | |
|---|---|
| α-pinene | 1.9 to 2.0% |
| α-terpinene | 1.2 to 2.7% |
| limnonene | 6.0 to 27.6% |
| ρ-cymene | 0.5 to 1.2% |
| 1,8 cineole | 2.5 to 25.6% |
| γ-terpinene | 2.8 to 6.5% |
| terpin-4-ol | 4.0 to 20.0% |
| γ-terpineol | 1.0 to 2.2% |
| neral | 2.2 to 9.2% |
| geranial | 1.5 to 11.5% |
| thymol | 0.3 to 2.0% |
| eugenol | 0.5 to 3.5% |
| butylated hydroxytoluene | 0.3 to 0.5% |
| Balance (insignificant variations | |
| in natural ingredients made up as | |
| required with ethanol) | |

3. A bactericidal preparation according to claim 1, being an ointment having the following ranges of composition by weight:

| | |
|---|---|
| Triclosan | 0.3 to 2.0% |
| Allantoin | 0.1 to 2.5% |
| Petrolatum | 40.0 to 70.0% |
| α-pinene | 1.9 to 2.0% |
| β-pinene | 1.2 to 2.7% |
| Myrcene | 0.5 to 1.2% |
| α-phellandrene | 0.3 to 0.4% |
| α-terpinene | 2.7 to 4.2% |
| limonene | 6.0 to 27.6% |
| β-phellandrene | 0.2 to 0.6% |
| 1,8 cineole | 2.5 to 25.6% |
| γ-terpinene | 2.8 to 6.5% |
| terpinolene | 1.0 to 4.0% |
| terpin-4-ol | 4.0 to 20.0% |
| α-terpineol | 1.0 to 2.2% |
| neral | 2.2 to 9.2% |
| geranial | 1.5 to 11.5% |
| thymol | 0.3 to 2.0% |
| eugenol | 0.5 to 3.5% |
| butylated hydroxytoluene | 0.3 to 0.5% |

4. A bactericidal preparation according to claim 2, being a solution have the following composition by weight:

| | |
|---|---|
| α-pinene | 1.9% |
| α-terpinene | 1.2% |
| limonene | 25.5% |
| ρ-cymene | 1.0% |
| 1,8 cineole | 15.6% |
| γ-terpinene | 2.8% |
| terpin-4-ol | 20.0% |
| γ-terpineol | 2.2% |
| neral | 9.2% |
| geranial | 10.9% |
| thymol | 1.0% |
| eugenol | 2.4% |
| butylated hydroxytoluene | 0.5% |
| Balance (insignificant variations | |
| in natural ingredients made up as | |
| required with ethanol) | |

5. A bactericidal preparation according to claim 3, being an ointment having the following composition by weight:

| | |
|---|---|
| Triclosan | 1.0% |
| Allantoin | 1.0% |
| Petrolatum | 68.0% |
| α-pinene | 2.0% |
| β-pinene | 1.4% |
| Myrcene | 0.7% |
| α-phellandrene | 0.3% |
| α-terpinene | 2.7% |
| limonene | 27.6% |
| β-phellandrene | 0.2% |
| 1,8 cineole | 23.2% |
| γ-terpinene | 6.5% |
| terpinolene | 1.0% |
| terpin-4-ol | 10.4% |
| α-terpineol | 1.0% |
| neral | 6.3% |
| geranial | 7.5% |
| thymol | 0.6% |
| eugenol | 2.3% |
| butylated hydroxytoluene | 0.3% |

6. A bactericidal composition according to any one of claims 2 or 4, wherein undenatured ethanol is added at varying levels, those levels being between 49.5% w-w and 97.4% w-w.

7. A bactericidal composition according to any one of the preceding claims wherein the solution, ointment or cream the activity thereof is achieved by means of absorption through the skin.

8. A bactericidal composition according to any one of the preceding claims, wherein the preparation acts on pathogens present in a nonspecific way by interrupting the metabolic uptake in the cell of pathogenic organisms and bursting the cell membrane.
